SEQUENCE LISTING (36 sequences)

SEQ ID NO: 1 - protein, 252 aa, Enterococcus faecalis
MKWMIVEKAEELANVSYQLLKQEIVRHPEGLTIGLATGSSPLGVYEEWRKDRVDCRHVTTVNLDEYVGLSPDHPHSYHTFMQEHLFDAVDFKESYVPIGSTADPREESDRYEALVRQLGIDIQLLGIGSNGHIAFNEPGTPFDAKTHVTKLTESTRQANQRFFDRLEDVPTEAITMGIGTIMEAKKILLVASSERKAEAIRDMMEGPATTDCPATILQRHADVMVVLDEEAASLLSDEAKRTGRAAYTNFMK

SEQ ID NO: 2 - DNA, 702 bp, Enterococcus faecalis
atgcagatcatccgtgttgctaacgctgaagaaggtggtaaaaaagctttcgaactgatcaaagaaggtatgaacaacggtgctaaagttctgggtctggctaccggttctaccccggaaaccctgtacaaagaaatgaccgcttctgacatcgacttcaccgaaatgacctctgttaacctggacgaatacgttggtctgggtggtgaagacgaacagtcttaccgttacttcatgaacaaacacctgttcgacaaaaaaccgttcaaagaaaccttcgttccgaacggtaaagctgaagacctggacgctgcttctgctgaatacgaaaaaatcatcgacgctcacccggttgacatccagatcctgggtatcggtcagaacggccatatcggcttcaacgaaccgggcaccccgctggactctctgacccacgttgttgaactgaccgaatctaccatcaacgctaacaaacgttacttcgacaaagttgaagacgttccgacccgtgctgtttctatgggtatcggttctatcatgaaaggtaaaaaaatgatcctgatggcttacggtgaagctaaagctgaagctatcaaaggtatgatcgacggtccggttaccaccgacatgccggcttctgctctgcagaaccaccaggacgttgttgttatcatcgacgacgctgctgcttctaaactgtaa

SEQ ID NO: 3 - protein, 252 aa, Exiguobacterium sibiricum; identical to SEQ ID NO: 1
  Es1 Variant

SEQ ID NO: 4 - DNA, 759 bp, Exiguobacterium sibiricum
  Es1 Variant
atgaaatggatgatcgttgaaaaagctgaagaactggctaacgtttcttaccagctgctgaaacaggaaatcgttcgtcacccggaaggtctgaccatcggtctggctaccggttcttctccgctgggtgtttacgaagaatggcgtaaagaccgtgttgactgccgtcacgttaccaccgttaacctggacgaatacgttggtctgtctccggaccacccgcactcttaccacaccttcatgcaggaacacctgttcgacgctgttgacttcaaagaatcttacgttccgatcggttctaccgctgacccgcgtgaagaatctgaccgttacgaagctctggttcgtcagctgggtatcgacatccagctgctgggtatcggttctaacggccatatcgcgttcaacgaaccgggcaccccgttcgacgctaaaacccacgttaccaaactgaccgaatctacccgtcaggctaaccagcgtttcttcgaccgtctggaagacgttccgaccgaagctatcactatgggtatcggtaccatcatggaagctaaaaaaatcctgctggttgcttcttctgaacgtaaagctgaagctatccgtgacatgatggaaggtccggctaccaccgactgcccggctaccatcctgcagcgtcacgctgacgttatggttgttctggacgaagaagctgcttctctgctgtctgacgaagctaaacgtaccggtcgtgctgcttacaccaacttcatgaaataa

SEQ ID NO: 5 - protein, 598 aa, Exiguobacterium sibiricum
  Es2 Variant
MCGIVGMIGQVNTKEILLKGLEKLEYRGYDSAGLAFVNDGVQVHKEVGRIAALREVVPAEADGLVGIGHTRWATHGVPSVPNAHPHQSASSRFTLVHNGVIENDEQLKAELNVDLLSDTDTEVIVQMIEKNFAETNDVVEAFRQTLRVLHGSYALALIDAENPDVLYVAKNKSPLLVGLGDGTFNVVASDAMAMLQVTDQFVELHDGEMIILTRDSVTIQDLDGNVKEREAYTAEIDASDIEKGTYAHYMLKEMDEQPAVIRNIVQKYQNESGEITLDQSVRDLVLGRDRVYIIGCGTSYHAGLIGKQLIEQIAGIPTEVHISSEFGYNMPLLTEKPLFLFLSQSGETADSRAVLVEAKKLGHPALTITNVAGSTLSREANATLLLHAGPEIAVASTKAYTAQIAVLAVLAFDLAQAKGVDVNFDLMKELGKISSAMESVMSQKERFQEIASEYLSESRNAFFIGRGQDAYVGMEGALKLKEISYIQAEGYAGGELKHGPIALIEDNTPVIALVTQPHVHLNNRGNVKEVVARGANACVIAAEGLELPTDAFVIPAVEPLLSPLLSVLPLQLISYYAALGRDCDVDKPRNLAKSVTVE

SEQ ID NO: 6 - DNA, 1797 bp, Exiguobacterium sibiricum
  Es2 Variant
atgtgcggtatcgttggtatgatcggtcaggttaacaccaaagaaatcctgctgaaaggtctggaaaaactggaataccgtggttacgactctgctggtctggctttcgttaacgacggtgttcaggttcacaaagaagttggtcgtatcgctgctctgcgtgaagttgttccggctgaagctgacggtctggttggtatcggtcacacccgttgggctacccacggtgttccgtctgttccgaacgctcacccgcaccagtctgcttcttctcgtttcaccctggttcacaacggtgttatcgaaaacgacgaacagctgaaagctgaactgaacgttgacctgctgtctgacaccgacaccgaagttatcgttcagatgatcgaaaaaaacttcgctgaaaccaacgatgttgttgaagcgttccgtcagaccctccgtgttctgcacggttcttacgctctggctctgatcgacgctgaaaacccggacgttctgtacgttgctaaaaacaaatctccgctgctggttggtctgggtgacggtaccttcaacgttgttgcttctgacgctatggctatgctgcaggttaccgaccagttcgttgaactgcacgacggtgaaatgatcatcctgacccgtgactctgttaccatccaggacctggacggtaacgttaaagaacgtgaagcttacaccgctgaaatcgacgcttctgacatcgaaaaaggtacctacgctcactacatgctgaaagaaatggacgaacagccggctgttatccgtaacatcgttcagaaataccagaacgaatctggtgaaatcaccctggaccagtctgttcgtgacctggttctgggtagggaccgtgtatacatcatcggctgcggtacctcttaccacgctggtctgatcggtaaacagctgatcgaacagatcgctggtatcccgaccgaagttcacatctcttctgaattcggttacaacatgccgctgctgaccgaaaaaccgctgttcctgttcctgtctcagtctggtgaaaccgctgactctcgtgctgttctggttgaagctaaaaaactgggtcacccggctctgaccatcaccaacgttgctggttctaccctgtctcgtgaagctaacgctaccctgctgctgcacgctggtccggaaatcgctgttgcttctaccaaagcttacaccgctcagatcgctgttctggctgttctggctttcgacctggctcaggctaaaggtgttgacgttaacttcgacctgatgaaagaactgggtaaaatctcttctgctatggaatctgttatgtctcagaaagaacgtttccaggaaatcgcttctgaatacctgtctgaatctcgtaacgctttcttcatcggtcgtggtcaggacgcttacgttggtatggaaggtgctctgaaactgaaagaaatctcttacatccaggctgaaggttacgctggtggtgaactgaaacacggtccgatcgctctgatcgaagacaacaccccggttatcgctctggttacccagccgcacgttcacctgaacaaccgtggtaacgttaaagaagttgttgctcgtggtgctaacgcttgcgttatcgctgctgaaggtctggaactgccgaccgacgctttcgttatcccggctgttgaaccgctgctgtctccgctgctgtctgttctgccgctgcagctgatctcttactacgctgctctgggtcgtgactgcgacgttgacaaaccgcgtaacctggctaaatctgttaccgttgaataa

SEQ ID NO: 7 - protein, 253 aa, Clostridium nexile
MGKQKKEKKGMKIIKTADYNEMSRKAANLIGAQVIMKPNCILGLATGSSPIGTYKELIKRCEEGDLDFSQVKSVNLDEYKGLPRDNDQSYYYFMNHNLFDHINIDKANTHVPNGMEPDAAKECANYEELIKSLGGVDLQLLGLGHNGHIGFNEPAEEFDKVTHCVDLQESTIEANKRFFESADDVPRQAYTMGIGTIMSAKKIVVVVSGEDKADIVKRAFSGPVTPSVPASILQMHPDVTVICDAAAYSKVEA

SEQ ID NO: 8 - DNA, 762 bp, Clostridium nexile
atgggtaaacagaaaaaagaaaaaaaaggtatgaaaatcatcaaaaccgctgactacaacgaaatgtctcgtaaagctgctaacctgatcggtgctcaggttatcatgaaaccgaactgcatcctgggtctggctaccggttcttctccgatcggtacctacaaagaactgatcaaacgttgcgaagaaggtgacctggacttctctcaggttaaatctgttaacctggacgaatacaaaggtctgccgcgtgacaacgaccagtcttactactacttcatgaaccacaacctgttcgaccacatcaacatcgacaaagctaacacccacgttccgaacggtatggaaccggacgctgctaaagaatgcgctaactacgaagaactgatcaaatctctgggtggtgttgacctgcagctgctgggtctgggtcacaacggccatatcggcttcaacgaaccggcggaagaattcgacaaagttacccactgcgttgacctgcaggaatctaccatcgaagctaacaaacgtttcttcgaatctgctgacgacgttccgcgtcaggcttacactatgggtatcggtaccatcatgtctgctaaaaaaatcgttgttgttgtttctggtgaagacaaagctgacatcgttaaacgtgctttctctggtccggttaccccgtctgttccggcttctatcctgcagatgcacccggacgttaccgttatctgcgacgctgctgcttactctaaagttgaagcttaa

SEQ ID NO: 9 - protein, 658 aa, Dyadobacter fermentans
MSQQTVSPQTILMQASSNGKGTTGTIGQPITYEKIPTQIFADSKEASYAVAKEISDLIRQKQKEGKPCVLGLATGSSPKTVYAILVRMHREEGLSFKNVISFNLDEYYQMEPDSIYSYHRFMKEQLFDHVDIPKENYFLPDGTVPAALLRDYCASYENKINAVGGLDFQLLGIGGNGHIGFNEPGSLINSHTRLITLDHSTRAAASMEFGGLHNVPRKAITLGVAPILNARRVVLLAWGERKAQVIKGAVEGPVTELNPASYLQAHADVSFVVDESAASELTRIKTPWAVDSVIWDNKMIKKAVTHLSQTLKKPILKLTDKDYNDNGMSDLLAQYGAGYEININVFNQLQHTITGWPGGKPNADDTHRPERAQPAKKRVLIFSPHPDDDIISMGGTFQRLVDQGHEVHVAYQTSGNIAVADDEALRFIDFVVDFNTGFDIESPNANKLFLDAKEFLRHTKNSEIDSPEVRKVKGLIRRGEAKATCRFVGIPTSQAHFLDMPFYETGTVQKKPIGEEDIKIIENLIEEVKPHQIYAAGDFADPHGTHKVCWDAIEAALQRSKHKNFMKDCWVWLYRGAWAEWDIYEIEMAVPMSPDQVLKKRQGIFKHQSQKDGVVFQGEDSREFWQRAEERNRGTAQLYDSLGLAEYEAMEAFVRWKF

SEQ ID NO: 10 - DNA, 1977 bp, Dyadobacter fermentans
atgtctcagcaaaccgtaagcccgcagaccatcctgatgcaggcttcttctaacggtaaaggtaccaccggtaccatcggtcagccgatcacctacgaaaaaatcccgacccagatcttcgctgactctaaagaagcttcttacgctgttgctaaagaaatctctgacctgatccgtcagaaacagaaagaaggtaaaccgtgcgttctgggtctggctaccggttcttctcccaagaccgtatacgcaatcctggttcgtatgcaccgtgaagaaggtctgtctttcaaaaacgttatctctttcaacctggacgaatactaccagatggaaccggactctatctactcttaccaccgtttcatgaaagaacagctgttcgaccacgttgacatcccgaaagaaaactacttcctgccggacggtaccgttccggctgctctgctgcgtgactactgcgcttcttacgaaaacaaaatcaacgctgttggtggtctggacttccagctgctgggtatcggtggtaacggccatatcggcttcaacgaaccgggctctctgatcaactctcacacccgtctgatcaccctggaccactctacccgtgctgctgcttctatggaattcggtggtctgcacaacgttccgcgtaaagctatcaccctgggtgttgctccgatcctgaacgctcgtcgtgttgttctgctggcttggggtgaacgtaaagctcaggttatcaaaggtgctgttgaaggtccggttaccgaactgaacccggcttcttacctgcaggctcacgctgacgtttctttcgttgttgacgaatctgctgcttctgaactgacccgtatcaaaaccccgtgggctgttgactctgttatctgggacaacaaaatgatcaaaaaagctgttacccacctgtctcagaccctgaaaaaaccgatcctgaaactgaccgacaaagactacaacgacaacggtatgtctgacctgctggctcagtacggtgctggttacgaaataaatatcaacgtgttcaaccaactgcagcacaccatcaccggttggccgggtggtaaaccgaacgctgacgacacccaccgtccggaacgtgctcagccggctaaaaaacgtgttctgatcttctctccgcacccggacgacgacatcatctctatgggtggtaccttccagcgtctggttgaccagggtcacgaagttcacgttgcttaccagacctctggtaacatcgctgttgctgacgacgaagctctgcgttttatcgacttcgttgttgactttaacaccggcttcgacatcgaatcgccgaacgctaacaaactgttcctggacgctaaagaattcctgcgtcacaccaaaaactctgaaatcgactctccggaagttcgtaaagttaaaggtctgatccgtcgtggtgaagctaaagctacctgccgtttcgttggtatcccgacctctcaggctcacttcctggacatgccgttctacgaaaccggtaccgttcagaaaaaaccgatcggtgaagaagacatcaaaatcatcgaaaacctgatcgaagaagttaaaccgcaccagatctacgctgctggtgacttcgctgacccgcacggtacccacaaagtttgctgggacgctatcgaagctgctctgcagcgttctaaacacaaaaacttcatgaaagactgctgggtttggctgtaccgtggtgcttgggctgaatgggacatctacgaaatcgaaatggctgttccgatgtctccggaccaggttctgaaaaaacgtcagggtatcttcaaacaccagtctcagaaagacggcgttgtattccagggcgaagactctcgtgaattctggcagcgtgctgaagaacgtaaccgtggtaccgctcagctgtacgactctctgggtctggctgaatacgaagctatggaagctttcgttcgttggaaattctaa

SEQ ID NO: 11 - protein, 249 aa, Pedobacter heparinus
  Ph1 variant
MTIKMETSVTNNPVELGKAAGKAAAALIRETIAKNGKANIILATGASQFETLNQLISEEIDWSKVTMFHLDEYIGLSESAPASFRKYLKERFLEKVSPLKAAYLVNGETDPEAECQRLGEIITQNPIDVALVGIGENGHLAFNDPPADFETKRPYLVVNLDEQCRRQQFGEGWFKTIEEVPTQAISMSVQQILKSKHIICSVPDSRKAQAVKDSLEQPVSNQYPSSILQLHPDCRFFFDKASAELLSEA

SEQ ID NO: 12 - DNA, 750 bp, Pedobacter heparinus
  Ph1 variant
atgaccatcaaaatggaaacctctgttaccaacaacccggttgaactgggtaaagctgctggtaaagctgctgctgctctgatccgtgaaaccatcgctaaaaacggtaaagctaacatcatcctggctaccggtgcttctcagttcgaaaccctgaaccagctgatctctgaagaaatcgactggtctaaagttaccatgttccacctggacgaatacatcggtctgtctgaatctgctccagcgagcttccgtaaatacctcaaagaacgtttcctggaaaaagtttctccgctgaaagctgcttacctggttaacggtgaaaccgacccggaagctgaatgccagcgtctgggtgaaatcatcacccagaacccgatcgacgttgctctggttggtatcggtgaaaacggccatctggcgttcaacgacccgccagctgacttcgaaaccaaacgtccgtacctggttgttaacctggacgaacagtgccgtcgtcagcagttcggtgaaggttggttcaaaaccatcgaagaagttccgacccaggctatctctatgtctgttcagcagatcctgaaatctaaacacatcatctgctctgttccggactctcgtaaagctcaggctgttaaagactctctggagcaaccggtaagcaaccagtacccgtcttctatcctgcagctgcacccggactgccgtttcttcttcgacaaagcttctgctgaactgctgtctgaagcttaa

SEQ ID NO: 13 - protein, 254 aa, Pedobacter heparinus
  Ph2 variant
MIKEFLKDKLKVKVYNTRGEMGTSAADLAAQCLRELLQHKDEVNIIFAAAASQNEFLEAIAVEKDIAWNRVNAFHMDEYTGLPVTHPQRFGNFLNKKIFSKLPLKKVYYLNEDGNDTEAESRRYAALLEKMPPDITFMGIGENTHLAFNDPHVADFNDPVLVKIIDLDEPCKVQQVHDGCFPTVAEVPSLAYTLTIPALLQSKYIFCMVPGKNKAQAVKYTLNESITAKYPSTSLRTHSDATLFLDQDSASLIL

SEQ ID NO: 14 - DNA, 765 bp, Pedobacter heparinus
  Ph2 variant
atgatcaaagaattcctgaaagacaaactgaaagttaaagtttacaacacccgtggtgaaatgggtacctctgctgctgacctggctgctcagtgcctgcgtgaactgctgcagcacaaagacgaagttaacatcatcttcgctgctgctgcttctcagaacgaattcctggaagctatcgctgttgaaaaagacatcgcttggaaccgtgttaacgctttccacatggacgaatacaccggtctgccagttacccatccgcagaggttcggtaacttcctcaacaaaaaaatcttctctaaactgccgctgaaaaaagtttactacctgaacgaagacggtaacgacaccgaagctgaatctcgtcgttacgctgctctgctggaaaaaatgccgccggacatcaccttcatgggtatcggtgaaaacacccacctggctttcaacgacccgcacgttgctgacttcaacgacccggttctggttaaaatcatcgacctggacgaaccgtgcaaagttcagcaggttcacgacggttgcttcccgaccgttgctgaagttccgtctctggcttacaccctgaccatcccggctctgctgcagtctaaatacatcttctgcatggttccgggtaaaaacaaagctcaggctgttaaatacaccctgaacgaatctatcaccgctaaatacccgtctacctctctgcgtacccactctgacgctaccctgttcctggaccaggactctgcttctctgatcctgtaa

SEQ ID NO: 15 - protein, 639 aa, Pedobacter heparinus
  Ph3 variant
MARLNLLEETRFEKLPVSVFENPKEASLSVAHRIGNLIKEKQKNNAQAVLGLATGATPIAVYAELVRMHREEGLSFKNVVTFNLDEYYPMQPNAAQSYVTFMNENLFDHIDIDKKNVNIPDGTLSLEEIPAFCLNYEKKIGDLGGLDIQILGIGRTGHIGFNEPGSAPNSGTRLVTLDDLTRRDAARDFGGKSFVPTKAITMGVGTIFKAREIILMAWNKKKAPIIKKTVEGEISSEVPATYLQLSDNVEFILDKDAASLLTRFDTPWLVKDCIWDEKLTRKAVIWLANTLKKPVLKLTEDDYNNNGMAQLAVEQGPVYNINIHIFNKLQHTITGWPGGKPNADDSQRPERAEPAKKRSIIFSPHPDDDVISMGGTFIRLVDQQQDVHVAYQTSGNTAVWDDDALRFVEFNIDFSEKMGLETAELKKLYQNMRSFIEQKKPNQIDTPEIQTVKGLIRKGEAIAGARYCGLADDHIHFMALPFYESGKSQKNPVTDADVVLTMELLQKVKPHQVFAAGDFEDPHGTHIVCFNIILEALRRLSKTEAWVKDCWLWMYRGAWQEFETYEIEMAVPLSPQEVERKKFAIFKHQSQKDRAVFPGDDAREFWQRAEDRNRETAQAYDSLGLAEYEAMEAFVRYKF

SEQ ID NO: 16 - DNA, 1920 bp, Pedobacter heparinus
  Ph3 variant
atggctcgtctgaacctgctggaagaaacccgtttcgaaaaactgccggtttctgttttcgaaaacccgaaagaagcttctctgtctgttgctcaccgtatcggtaacctgatcaaagaaaaacagaaaaacaacgctcaggctgttctgggtctggctaccggtgctaccccaatagctgtgtacgcagaactggttcgtatgcaccgtgaagaaggtctgtctttcaaaaacgttgttaccttcaacctggacgaatactacccgatgcagccgaacgctgctcagtcttacgttaccttcatgaacgaaaacctgttcgaccacatcgacatcgacaaaaaaaacgttaacatcccggacggtaccctgtctctggaagaaatcccggctttctgcctgaactacgaaaaaaaaatcggtgacctgggtggtctggacatccagatcctgggtatcggtcgtaccggtcatataggcttcaacgaaccgggctctgctccgaactctggtacccgtctggttaccctggacgacctgacccgtcgtgacgctgctcgtgacttcggtggtaaatctttcgttccgaccaaagctatcactatgggtgttggtaccatcttcaaagctcgtgaaatcatcctgatggcttggaacaaaaaaaaagctccgatcatcaaaaaaaccgttgaaggtgaaatctcttctgaagttccggctacctacctgcagctgtctgacaacgttgaattcatcctggacaaagacgctgcttctctgctgacccgtttcgacaccccgtggctggttaaagactgcatctgggacgaaaaactgacccgtaaagctgttatctggctggctaacaccctgaaaaaaccggttctgaaactgaccgaagacgactacaacaacaacggtatggctcagctggctgttgaacagggtccggtttacaacatcaacatccacatcttcaacaaactgcagcacaccatcaccggttggccgggtggtaaaccgaacgctgacgactctcagcgtccggaacgtgctgaaccggctaaaaaacgttctatcatcttctctccgcacccggacgacgacgttatctctatgggtggtaccttcatccgtctggttgaccagcagcaggacgttcacgttgcttaccagacctctggtaacaccgctgtttgggacgacgacgctctgcgtttcgttgaattcaacatcgacttctctgaaaaaatgggtctggaaaccgctgaactgaaaaaactgtaccagaacatgcgtagcttcatcgaacagaagaaaccgaaccagatcgacaccccggaaatccagaccgttaaaggtctgatccgtaaaggtgaagctatcgctggtgctcgttactgcggtctggctgacgaccacatccacttcatggctctgccgttctacgaatctggtaaatctcagaaaaacccggttaccgacgctgacgttgttctgactatggaactgctgcagaaagttaaaccgcaccaggttttcgctgctggtgacttcgaagacccgcacggtacccacatcgtttgcttcaacatcatcctggaagctctgcgtcgtctgtctaaaaccgaagcttgggttaaagactgctggctgtggatgtaccgtggtgcttggcaggaattcgaaacctacgaaatcgaaatggctgttccgctgtctccgcaggaagttgaacgtaaaaaattcgctatcttcaaacaccagtctcagaaagaccgtgctgttttcccgggtgacgacgctcgtgaattctggcagcgtgctgaagaccgtaaccgtgaaaccgctcaggcttacgactctctgggtctggctgaatacgaagctatggaagctttcgttcgttacaaattctaa

SEQ ID NO: 17 - protein, 172 aa, Granulicella tundricola
MRTGSHGWSDHGQALRKQAIVDALSLGVAKHHPRDLRLFAAVLEKNNFAGQDIAQIAFEQLSSRFDQYLGRLYREKGDKQRGLILFDKSSTERRIQTLARDFKHTGHSFGITRNYAEVPVFLDSRASRLIQLADLVAYAIFRHHEHGDSTYYNACSHCFDAEGGVVHGLYVR

SEQ ID NO: 18 - DNA, 519 bp, Granulicella tundricola
atgcgtaccggttctcacggttggtctgaccacggtcaggctctgcgtaaacaggctatcgttgacgctctgtctctgggtgttgctaaacaccacccgcgtgacctgcgtctgttcgctgctgttctggaaaaaaacaacttcgctggtcaggacatcgctcagatcgctttcgaacagctgtcttctcgtttcgaccagtacctgggtcgtctgtaccgtgaaaaaggtgacaaacagcgtggtctgatcctgttcgacaaatcttctaccgaacgtcgtatccagaccctggctcgtgacttcaaacacaccggtcactctttcggtatcaccagaaactacgctgaagttccggtattcctggacagccgtgcttctcgtctgatccagctggctgacctggttgcttacgctatcttccgtcaccacgaacacggtgactctacctactacaacgcttgctctcactgcttcgacgctgaaggtggtgttgttcacggtctgtacgttcgttaa

SEQ ID NO: 19 - protein, 629 aa, Terriglobus roseus
MCGIVGYIGARPAVPVIMDGLRRLEYRGYDSAGIALGGADTGLSIRRASGKLRNLESSLSLNPLQGDYGIGHTRWATHGRPSETNAHPHRDCTGSLVVVHNGIVENYCELQDQLKAKGHFFLTETDTEIIAHLIEQEYLDLRERNDGDAAPSLEKAVSQAVKKLTGAFAIGVISTDDPGKLVAARSGPPAVVGFGDGEYFLASDVPGILQYTRDIVFLQDGDVAVLTRSGISFSDFHGKPLKRSPQHITWDPIQAEKNGYAHFMLKEIYEQPRAVRDTLRGRISAASGHIELPGLDLSHDEIVHAERILIASCGTSWHAGLVGKFLLERMVRIPVEVDYASEFRYRRPIMRSRDIGLLISQSGETADTLAAQLEMIAGDIPTLSICNVVDAALTRKARGSITTNAGPEIGVASTKAFTAQIAALLTLSLYFAQCRKTMPQEAVQSILRELEVLPKAMEEALRTSAPVCARLAKIFSNATDVLFLGRGIHYPIALEGALKLKEISYIHAEGYPAGEMKHGPNALIDETLPVVCLATKDGDDPDSVLKYEKTLSNIQEVTARGGRVIAIVVEGDTAVSSLVEHTITIPRASEALLPILETVPLQLLAYFIAVEKGCDVDQPRNLAKSVTVE

SEQ ID NO: 20 - DNA, 1890 bp, Terriglobus roseus
atgtgcggtatcgttggttacatcggtgctcgtccggctgttccggttatcatggacggtctgcgtcgtctggaataccgtggttacgactctgctggtatcgctctgggtggtgctgacaccggtctgtctatccgtcgtgcttctggtaaactgcgtaacctggaatcttctctgtctctgaacccgctgcagggtgactacggtatcggtcacacccgttgggctacccacggtcgtccgtctgaaaccaacgctcacccgcaccgtgactgcaccggttctctggttgttgttcacaacggtatcgttgaaaactactgcgaactgcaggaccagctgaaagctaaaggtcacttcttcctgaccgaaaccgacaccgaaatcatcgctcacctgatcgaacaggaatacctggacctgcgtgaacgtaacgacggtgacgctgctccgtctctggaaaaagctgtttctcaggctgttaaaaaactgaccggtgctttcgctatcggtgttatctctaccgacgacccgggtaaactggttgctgctcgttctggtccgccggctgttgttggtttcggtgacggtgaatacttcctggcttctgacgttccgggtatcctgcagtacactcgtgacatcgtcttcctgcaagacggtgacgttgctgttctgacccgttctggtatctctttctctgacttccacggtaaaccgctgaaacgttctccgcagcacatcacctgggacccgatccaggctgaaaaaaacggttacgctcacttcatgctgaaagaaatctacgaacagccgcgtgctgttcgtgacaccctgcgtggtcgtatctctgctgcttctggtcacatcgaactgccgggtctggacctgtctcacgacgaaatcgttcacgctgaacgtatcctgatcgcttcttgcggtacctcttggcacgctggtctggttggtaaattcctgctggaacgtatggttcgtatcccggttgaagttgactacgcttctgaattccgttaccgtcgtccgatcatgcgttctcgtgacatcggtctgctgatctctcagtctggtgaaaccgctgacaccctggctgctcagctggaaatgatcgctggtgacatcccgaccctgtctatctgcaacgttgttgacgctgctctgacccgtaaagctcgtggttctatcaccaccaacgctggtccggaaatcggtgttgcttctactaaggcgttcaccgctcagatagctgctctgctgaccctgtctctgtacttcgctcagtgccgtaaaaccatgccgcaggaagctgttcagtctatcctgcgtgaactggaagttctgccgaaagctatggaagaagctctgcgtacctctgctccggtttgcgctcgtctggctaaaatcttctctaacgctaccgacgttctgttcctgggtcgtggtatccactacccgatcgctctggaaggtgctctgaaactgaaagaaatctcttacatccacgctgaaggttacccggctggtgaaatgaaacacggtccgaacgctctgatcgacgaaaccctgccggttgtttgcctggctaccaaagacggtgacgacccggactctgttctgaaatacgaaaaaaccctgtctaacatccaggaagttaccgctcgtggtggtcgtgttatcgctatcgttgttgaaggtgacaccgctgtttcttctctggttgaacacaccatcaccatcccgcgtgcttctgaagctctgctgccgatcctggaaaccgttccgctgcagctgctggcttacttcatcgctgttgaaaaaggttgcgacgttgaccagccgcgtaacctggctaaatctgttaccgttgaataa

SEQ ID NO: 21 - protein, 294 aa, Akkermansia muciniphila
MRAAPRKAVGRVEASVPWDFSRKYAAVHHSGTISVENARKRSKIGRVMKVETFETPQDAAKALAGEVAELIRTRAAEGKNVVLGLATGATPLPFYAELVRMHKEEGLSFANVISFNLDEYSGLDRDHPESYWYFMHTNLFNHIDIKPENINLPSGTVKDDEIAAHCAAYEQKIKDCGGIDLQILGIGRTGHIGFNEPGSDDTTVTRQVHLDELTRSDAAPAFGGIENVPTTAITMGVATIMGAREVALMAWGEKKASIVKKAVQGPVTVDVAASYLQKHPNAKFLLDKGAASLL

SEQ ID NO: 22 - DNA, 885 bp, Akkermansia muciniphila
atgcgtgctgctccgcgtaaagctgttggtcgtgttgaagcttctgttccgtgggacttctctcgtaaatacgctgctgttcaccactctggtaccatctctgttgaaaacgctcgtaaacgttctaaaatcggtcgtgttatgaaagttgaaaccttcgaaaccccgcaggacgctgctaaagctctggctggtgaagttgctgaactgatccgtacccgtgctgctgaaggtaaaaacgttgttctgggtctggctaccggtgctaccccgctgccgttctacgctgaactggttcgtatgcacaaagaagaaggtctgtctttcgctaacgttatctctttcaacctggacgaatactctggtctggaccgtgaccacccggaatcttactggtacttcatgcacaccaacctgttcaaccacatcgacatcaaaccggaaaacatcaacctgccgtctggtaccgttaaagacgacgaaatcgctgctcactgcgctgcttacgaacagaaaatcaaagactgcggtggtatcgacctgcagatcctgggtatcggtcgtaccggtcatataggcttcaacgaaccgggctctgacgacaccaccgttacccgtcaggttcacctggacgaactgacccgttctgacgctgcccctgcgttcggtggtatcgagaacgttccgaccaccgctatcactatgggtgttgctaccatcatgggtgctcgtgaagttgctctgatggcttggggtgaaaaaaaagcttctatcgttaaaaaagctgttcagggtccggttaccgttgacgttgctgcttcttacctgcagaaacacccgaacgctaaattcctgctggacaaaggtgctgcttctctgctgtaa

SEQ ID NO: 23 - protein, 270 aa, Bifidobacterium longum
  Bl1 variant
MPEIIIVKNEAEAGEIYGRCVADLIKAKPDAVLGLATGSSPLAAYQALAKIVKDEAIDVSGVRGFALDEYIGLPLTHPESYHATIHRTVVEPLGLDPAKVHVPGDVLNGTPLEDGDKVALAGPAYDRAIEAAGGIDVQILGIGTDGHVGFNEPGSSLASGTRVKTLAEQTRVDNARFFDNDINQVPTHCITQGIGTIMKARHLVLLAFGAGKAEAIEETVEGGLSAFCPASALQMHPHATIIVDEEAASRLRHKDYYRYAYTHKPAWQGI

SEQ ID NO: 24 - DNA, 813 bp, Bifidobacterium longum
  Bl1 variant
atgccggaaatcatcatcgttaaaaacgaagctgaagctggtgaaatctacggtcgttgcgttgctgacctgatcaaagctaaaccggacgctgttctgggtctggctaccggttcttctccgctggctgcttaccaggctctggctaaaatcgttaaagacgaagctatcgacgtttctggtgttcgtggtttcgctctggacgaatacatcggtctgccgctgacccacccggaatcttaccacgctaccatccaccgtaccgttgttgaaccgctgggtctggacccggctaaagttcacgttccgggtgacgttctgaacggtaccccgctggaagacggtgacaaagttgctctggctggtccggcttacgaccgtgctatcgaagctgctggtggtatcgacgttcagatcctgggtatcggtaccgacggtcatgtaggcttcaacgaaccgggctcttctctggcttctggtacccgtgttaaaaccctggctgaacagacccgtgttgacaatgcgaggttcttcgacaacgacatcaaccaggttccgacccactgcatcacccagggtatcggtaccatcatgaaagctcgtcacctggttctgctggctttcggtgctggtaaagctgaagctatcgaagaaaccgttgaaggtggtctgtctgctttctgcccggcttctgctctgcagatgcacccgcacgctaccatcatcgttgacgaagaagctgcttctcgtctgcgtcacaaagactactaccgttacgcttacacccacaaaccggcttggcagggtatctaa

SEQ ID NO: 25 - protein, 270 aa, Bifidobacterium longum; identical to SEQ ID NO: 23
  Bl2 variant

SEQ ID NO: 26 - DNA, 813 bp, Bifidobacterium longum; identical to SEQ ID NO: 24
  Bl2 variant

SEQ ID NO: 27 - protein, 242 aa, Myxobacterium xanthis
MRIRVFDSEREAAATCAQRIARAAAAHPSLVLGLPTGRTPLNVYRELVELFTRGGLDWAQVRTFNLDEFLGVSADDAGSFRAYMERHLFQHVNLSPAHIQFLDGAVEDAEAECARYEARLAEAGGLDLVLLGLGSNGHLAFNEPADGLRARCHRTRLSRQTREANLMLFGDDPSRVPMEALTLGMASILQARQALLLAFGEAKAEAVRGMVEGPVSPRCPASFLQLHPDVEVWLDPAAARAL

SEQ ID NO: 28 - DNA, 729 bp, Myxobacterium xanthis
atgcgtatccgtgttttcgactctgaacgtgaagctgctgctacctgcgctcagcgtatcgctcgtgctgctgctgctcacccgtctctggttctgggtctgccgaccggtcgtaccccgctgaacgtttaccgtgaactggttgaactgttcacccgtggtggtctggactgggctcaggttcgtaccttcaacctggacgaatttctcggtgtaagcgctgacgacgctggttctttccgtgcttacatggaacgtcacctgttccagcacgttaacctgtctccggctcacatccagttcctggacggtgctgttgaagacgctgaagctgaatgcgctcgttacgaagctcgtctggctgaagctggtggtctggacctggttctgctgggtctgggttctaacggtcacctggctttcaacgaaccggctgacggtctgcgtgctcgttgccaccgtacccgtctgtctcgtcagacccgtgaagctaacctgatgctgttcggtgacgacccgtctcgtgttccgatggaagctctgaccctgggtatggcttctatcctgcaggctcgtcaggctctgctgctggctttcggtgaagctaaagctgaagctgttcgtggtatggttgaaggtccggtttctccgcgttgcccagctagcttcctgcagctgcatccggacgttgaagtttggctggacccggctgctgctcgtgctctgtaa

SEQ ID NO: 29 - protein, 369 aa, Stackebrandtia nassauensis
  Sn1 variant
MSQQQAARSTTAGATDATAATPPGAGMAADIADQPEAFARLAETQSGAIADIAAEIARERPRFVMFTARGTSDHAALYAAYLTEIRLGIPAGLASPSAITVYGARPDLSEALVVGVSQSGGSPDIAEVLRVASESGARTLAVTNNPESPLAKQAGLNIDVSAGHEKAVAATKTYTAELLALFMLIEGIRAGDGKLAGDEAAAVAALPELARTVLADETPVQLAARYRFAERLVTTGRGYAYPTARETALKLMETSYLSALSFSGADLLHGPLAMADPDIPVLAIVGDGPGGNAMRDVIGRLGERRADVVSIGASDVEGAPIRIPVPAVDERLAPMLDILPLQRLALALALNRGENPDAPRGLRKVTETL

SEQ ID NO: 30 - DNA, 1110 bp, Stackebrandtia nassauensis
  Sn1 variant
atgtctcagcagcaggctgctcgttctaccaccgctggtgctaccgacgctaccgctgctaccccgccgggtgctggtatggctgctgacatcgctgaccagccggaagctttcgctcgtctggctgaaacccagtctggtgctatcgctgacatcgctgctgaaatcgctcgtgaacgtccgcgtttcgttatgttcaccgctcgtggtacctctgaccacgctgctctgtacgctgcttacctgaccgaaatccgtctgggtatcccggctggtctggcttctccgtctgctatcaccgtttacggtgctcgtccggacctgtctgaagctctggttgttggtgtttctcagtctggtggttctccggacatcgctgaagttctgcgtgttgcttctgaatctggtgctcgtaccctggctgttaccaacaacccggaatctccgctggctaaacaggctggtctgaacatcgacgtttctgctggtcacgaaaaagctgttgctgctaccaaaacctacaccgctgaactgctggctctgttcatgctgatcgaaggtatccgtgctggtgacggtaaactggctggtgacgaagctgctgctgttgctgctctgccggaactggctcgtaccgttctggctgacgaaaccccggttcagctggctgctcgttaccgtttcgctgaacgtctggttaccaccggtcgtggttacgcttacccgaccgctcgtgaaaccgctctgaaactgatggaaacctcttacctgtctgctctgtctttctctggtgctgacctgctgcacggtccgctggctatggctgacccggacatcccggttctggctatcgttggtgacggtccgggtggtaacgctatgcgtgacgttatcggtcgtctgggtgaacgtagggctgacgttgtaagcatcggtgcttctgacgttgaaggtgctccgatccgtatcccggttccggctgttgacgaacgtctggctccgatgctggacatcctgccgctgcagcgtctggctctggctctggctctgaaccgtggtgaaaacccggacgctccgcgtggtctgcgtaaagttaccgaaaccctgtaa

SEQ ID NO: 31 - protein, 289 aa, Stackebrandtia nassauensis
  Sn2 variant
MAIVDAEIARQPECWTAAAELAVEHAGLFPKAGERVAVVGCGTSWFMAQAVAGLREASGAGETDAFTASEFPDRRYDRLIALSRSGTTSEVVELLNRTDIPSLAIVGDGDTPAVAAADAAIAMPFASDQSVVMTSFATSVVALFRAQLGIDQAAAIADCRTALTAELPVDVAKLEQISFIGAGWTIGLANEAALKAREAATFWAESYPAMDYRHGPISIAGPNRATWELGPTGAGLRAEVEATGGAFVASSLDPLAELVLAQRLAVALARLRGLNPDSPRHLTRSVVLP

SEQ ID NO: 32 - DNA, 870 bp, Stackebrandtia nassauensis
  Sn2 variant
atggctatcgttgacgctgaaatcgctcgtcagccggaatgctggaccgctgctgctgaactggctgttgaacacgctggtctgttcccgaaagctggtgaacgtgttgctgttgttggttgcggtacctcttggttcatggctcaggctgttgctggtctgcgtgaagcttctggtgctggtgaaactgacgcgttcaccgcttctgagttcccggaccgtcgttacgaccgtctgatcgctctgtctcgttctggtaccacctctgaagttgttgaactgctgaaccgtaccgacatcccgtctctggctatcgttggtgacggtgacaccccggctgttgctgctgctgacgctgctatcgctatgccgttcgcttctgaccagtctgttgttatgacctctttcgctacctctgttgttgctctgttccgtgctcagctgggtatcgaccaggctgctgctatcgctgactgccgtaccgctctgaccgctgaactgccggttgacgttgctaaactggaacagatctctttcatcggtgctggttggaccatcggtctggctaacgaagctgctctgaaagctcgtgaagctgctaccttctgggctgaatcttacccggctatggactaccgtcacggtccgatctctatcgctggtccgaaccgtgctacctgggaactgggtccgaccggtgctggtctgcgtgctgaagttgaagctaccggtggtgctttcgttgcttcttctctggacccgctggctgaactggttctggctcagcgtctggctgttgctctggctcgtctgcgtggtctgaacccggactctccgcgtcacctgacccgttctgttgttctgccgtaa

SEQ ID NO: 33 - protein, 233 aa, Artificial Sequence
  Ef-Ser74Ala
MQIIRVANAEEGGKKAFELIKEGMNNGAKVLGLATGSTPETLYKEMTASDIDFTEMTSVNLDEYVGLGGEDEQSYRYFMNKHLFDKKPFKETFVPNGKAEDLDAASAEYEKIIDAHPVDIQILGIGQNGHIGFNEPGTPLDSLTHVVELTESTINANKRYFDKVEDVPTRAVSMGIGSIMKGKKMILMAYGEAKAEAIKGMIDGPVTTDMPASALQNHQDVVVIIDDAAASKL

SEQ ID NO: 34 - DNA, 702 bp, Artificial Sequence; identical to SEQ ID NO: 2
  Ef-Ser74Ala

SEQ ID NO: 35 - protein, 233 aa, Artificial Sequence; identical to SEQ ID NO: 33
  Ef-Thr144Ala

SEQ ID NO: 36 - DNA, 702 bp, Artificial Sequence; identical to SEQ ID NO: 2
  Ef-Thr144Ala

(19) United States
(12) Patent Application Publication
(10) Pub. No.: US 2024/0392278 A1
(43) Pub. Date: Nov. 28, 2024

(54) PROCESS FOR THE DIRECT ENZYMATIC CONVERSION OF AMINO SUGARS; ENZYME AND COMPOSITIONS FOR USE IN THE PROCESS

(71) Applicant: GLYCOMICS AND GLYCAN BIOENGINEERING RESEARCH CENTER (GGBRC), COLLEGE OF FOOD SCIENCE AND TECHNOLOGY,, Nanjing, Jiangsu (CN)

(72) Inventors: Josef VOGLMEIR, Jiangsu (CN); Li LIU, Jiangsu (CN)

(21) Appl. No.: 17/917,859

(22) PCT Filed: Mar. 30, 2021
(86) PCT No.: PCT/CN2021/083999

§ 371 Date: Oct. 7, 2022

(51) Int. Cl. C12N 9/78; C12P 3/00; C12P 19/02

(52) U.S. Cl. CPC C12N 9/78; C12P 3/00; C12P 19/02; C12Y 305/99006

(57) ABSTRACT

A process for converting amino sugar to fructose and ammonia using one enzyme. Also provided are compositions and enzymes for converting amino sugar to fructose and ammonia.

The present invention relates to a process for converting amino sugar to fructose and ammonia using one enzyme, an enzyme for converting amino sugar to fructose and ammonia, the use of an enzyme in the conversion of at least one amino sugar into fructose and a composition for converting amino sugar to fructose and ammonia.

Fructose and ammonia are very important compounds for a vast variety of applications. It is well known in the art, that both compounds are obtainable via the indirect enzymatic conversion of glucosamine.

Glucosamine is an amino sugar and one of the most abundant monosaccharides in nature. It is the main building block of the polysaccharides chitin and chitosan. Glucosamine can be produced by the hydrolysis of chitin-or chitosan-containing biomaterials, such as shellfish exoskeletons or fungi (Benavente, M., et al. (2015) J. Pharm. Pharmacol., 3, 20-26; Lv, Y. M., et al. (2017) Green Chem., 19, 527-535).

The prior art teaches the indirect enzymatic conversion of fructose and ammonia from glucosamine involving three different classes of enzymes:
(i) sugar kinases (EC 2.7.1.2), such as the enzymes of Sulfurisphaera tokodaii or Thermus caldophilus, transfer in a first step a phosphate group from the required co-factor adenosine triphosphate (ATP) to glucosamine, yielding glucosamine 6-phosphate (Nishimasu, H., et al. (2006) J. Bacteriol. 188, 2014-2019; Bae, J., et al. (2005) Biochem. Biophys. Res. Commun. 334, 754-763);
(ii) glucosamine-6-phosphate deaminases (EC 3.5.99.6), such as the enzymes of Thermococcus kodakarensis or Bacillus subtilis, catalyze the enzymatic deamination-isomerization of glucosamine 6-phosphate to fructose-6 phosphate and ammonia (Tanaka, T., et al. (2005) J. Bacteriol. 187, 7038-7044; Vincent, F., et al. (2005) J. Biol. Chem. 280, 19649-19655); and
(iii) sugar phosphatases (EC 3.1.3.23), such as the enzymes of Escherichia coli or Pseudomonas fluorescens, catalyze the hydrolysis of a phosphate group from fructose-6 phosphate to fructose (Lee, Y. P., et al. (1975) J. Biol. Chem. 250, 3729-3737; Maleki, S., et al. (2017) Appl. Environ. Microbiol. 83, e02361).

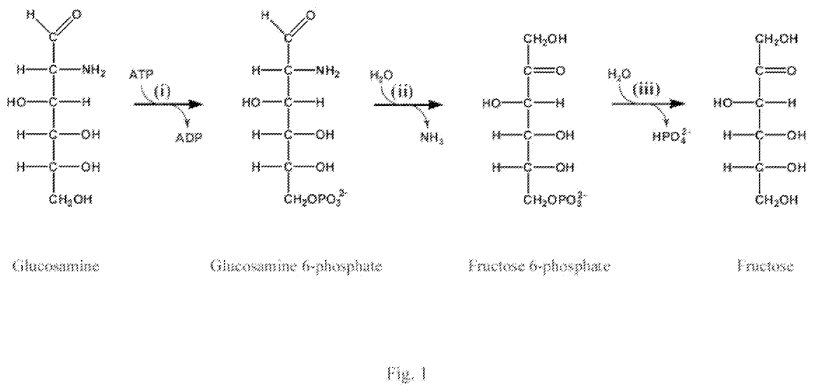

FIG. 1 schematically depicts an enzymatic process, in accordance with the state of the art, for converting glucosamine into fructose and ammonia.

A direct enzymatic conversion from amino sugar such as glucosamine to fructose and ammonia catalyzed by one enzyme would be desirable. Such a one-step process could simplify and speed up the process. Moreover, from an ecological point of view, saving natural resources and energy would be beneficial.

In fact, despite its extremely large abundance in nature, glucosamine has not yet been used efficiently as renewable carbon source, whereas the fermentation of the resulting fructose is an established and cost-effective process in the industrial production of ethanol. Furthermore, the direct production of ammonia from glucosamine catalyzed by the GPDA (glucosamine-6-phosphate deaminase) enzyme is a desirable alternative to the existing industrial ammonia production processes.

The major challenge faced by the inventors consisted in the use of glucosamine instead of glucosamine 6-phosphate as a substrate for the GPDA enzyme-catalyzed reaction.

Therefore it is the object of the present invention to provide a direct enzymatic conversion process for the preparation of fructose and ammonia from amino sugar.

This object is solved by the present invention.

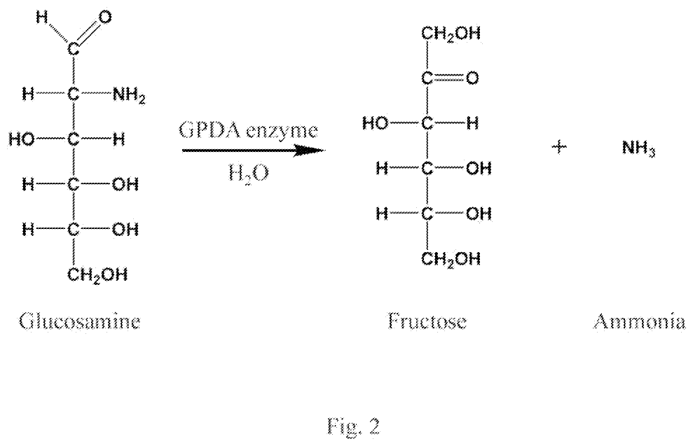

In a first aspect the present invention relates to a process for converting amino sugar to fructose and ammonia using one enzyme as schematically depicted in FIG. 2.

In a second aspect the present invention relates to an enzyme for converting amino sugar to fructose and ammonia.

In a further aspect, the present invention is directed to the use of an enzyme in the conversion of at least one amino sugar into fructose and ammonia.

In a further aspect the present invention is directed to a composition comprising ammonia and fructose obtained by a process for converting amino sugar to fructose and ammonia using one enzyme.

In another aspect the present invention relates to a composition for converting amino sugar to fructose and ammonia comprising
water;
an amino sugar, preferably an amino monosaccharide, more preferably a 2-amino-2-deoxysugar and even more preferably glucosamine;
a glucosamine-6-phosphate deaminase (GPDA enzyme), preferably glucosamine-6-phosphate deaminase (GPDA enzyme) with an amino acid sequence that is at least 80%, preferably at least 90%, more preferably at least 95% and even more preferably at least 98.5% identical with the sequence according to SEQ ID NO: 1.

It has surprisingly been found that the above objective can be achieved by the use of a single enzyme.

Preferably, said single enzyme is a hydrolase, more preferably said enzyme is glucosamine-6-phosphate deaminase (GPDA enzyme), which is suitable to efficiently convert amino sugars into fructose and ammonia in an one step process.

More specifically, it has been found that a process involving only one enzyme, preferably a hydrolase, more preferably a GPDA enzyme allows for the direct conversion of amino sugars to fructose and ammonia without the need for a phosphorylation step.

The present invention provides a process for converting amino sugar to fructose and ammonia using one enzyme.

Preferably, the process is a one-step process.

Optionally, the process of the present invention further comprises a step of hydrolyzing a biomaterial, preferably a biomaterial containing amino sugars, preferably containing amino polysaccharides, more preferably containing chitin and/or chitosan.

The amino sugar is an amino monosaccharide, preferably a 2-amino-2-deoxysugar and, more preferably, the amino sugar is glucosamine.

According to an embodiment of the present invention, the amino sugar is obtained from hydrolyzing a biomaterial, preferably a biomaterial containing amino sugars, preferably containing amino polysaccharides, more preferably chitin-or chitosan-containing biomaterials, such as shellfish exoskeletons, insects or fungi.

Suitable enzymes for use in the process of the present invention include hydrolase enzymes, preferably glucosamine-6-phosphate deaminase (GPDA enzyme), such as GPDA enzyme derived from Exiguobacterium sibiricum; Clostridium nexile; Dyadobacter fermentans; Pedobacter heparinus; Granulicella tundricola; Enterococcus faecalis; Terriglobus roseus; Akkermansia muciniphila; Bifidobacterium longum; Myxobacterium xanthis or Stackebrandtia nassauensis. Preferably, the GPDA enzyme is derived from Exiguobacterium sibiricum; Pedobacter heparinus; Granulicella tundricola; Enterococcus faecalis, Akkermansia muciniphila; Bifidobacterium longum; Myxoacterium xanthis or Stackebrandtia nassauensis. Most preferably, the GPDA enzyme is Ef-GPDA from Enterococcus faecalis.

Several enzymes may be derived from one and the same organism of origin, in other words, homologs derived from the same organism of origin are possible. Nevertheless, such homologs are ‘wild-type’ variants.

The enzyme may be selected from mutant variants or “wild-type” variants. In the context of the present application, the term “wild-type” refers to the typical form of an enzyme and/or gene as it occurs in nature.

Unless otherwise indicated, all enzymes referred to herein, are “wild-type” variants.

Most preferably, the GPDA enzyme is the wild-type form of Ef-GPDA from Enterococcus faecalis.

The enzyme, preferably hydrolase, more preferably glucosamine-6-phosphate deaminase (GPDA enzyme) may be in the form of an isolated polypeptide or a whole-cell biocatalyst.

In some embodiments the enzyme, preferably the hydrolase, more preferably glucosamine-6-phosphate deaminase (GPDA enzyme), is immobilized.

The process according to the present invention comprises a step of contacting the glucosamine with one enzyme in the presence of water.

The enzymatic conversion achieved by the process of the present invention is a deamination-isomerization reaction.

The process according to the present invention is carried out at a temperature in a range of from 4 to 50° C., preferably from 16 to 42° C., more preferably from 30to 42° C. and most preferably at 37° C.

The process according to the present invention is carried out at a pH in the range of from 4.0 to 10.0, preferably from 4.0 to 8.0, more preferably from 6.5 to 8.0and most preferably at a pH of 7.0.

The process according to the present invention comprises incubating the components for at least 3 hours, preferably at least 6 hours, more preferably at least 12 hours. Preferably the maximum duration of the step of incubating the components is 96 hours, more preferably 72 hours.

In the process according to the present invention at least 10%, preferably at least 15% of the amino sugar are converted to fructose and ammonia after 24 h of enzymatic conversion.

Moreover, the process according to the present invention comprises a step of providing a composition comprising:
water;
an amino sugar, preferably an amino monosaccharide, more preferably a 2-amino-2-deoxysugar and even more preferably glucosamine;
an enzyme, preferably glucosamine-6-phosphate deaminase (GPDA enzyme), more preferably the glucosamine-6-phosphate deaminase is the wild-type form of glucosamine-6-phosphate deaminase derived from Enterococcus faecalis (Ef-GPDA enzyme, wild-type) and/or the glucosamine-6-phosphate deaminase (GPDA enzyme) comprises an amino acid sequence that is at least 80%, preferably at least 90%, more preferably at least 95% and even more preferably at least 98.5% identical with the sequence according to SEQ ID NO: 1.

The composition according to the present invention may further comprise additives, for example, buffer solutions.

The concentration of the enzyme in the composition is in a range of from 0.2 g/L to 10 g/L.

The concentration of the amino sugar in the composition is in a range of from 1mM to 500 mM.

If present, the concentration of a pH buffer in the composition is in a range of from 0 to 200 mM.

The concentration of, optionally contained, further additives in the composition is in a range of from 0 to 5 mM. An example of a further additive is magnesium chloride (MgCl₂).

The composition according to the present invention does not comprise phosphates, neither in free nor conjugated form.

The present invention further relates to an enzyme for converting amino sugar, preferably amino monosaccharide, preferably 2-amino-2-deoxysugar and more preferably glucosamine to fructose and ammonia.

Specifically, the enzyme is a hydrolase, preferably glucosamine-6-phosphate deaminase (GPDA enzyme), more preferably glucosamine-6-phosphate deaminase (GPDA enzyme) comprising an amino acid sequence that is at least 80%, preferably at least 90%, more preferably at least 95% and even more preferably at least 98.5% identical with the sequence according to SEQ ID NO: 1.

Moreover, the requirements regarding the enzyme as already pointed out above, that is in the context of the process according to the present invention, are equally applicable to the enzyme as such and vice-versa.

In a further embodiment, the present invention is directed to the use of an enzyme (as detailed above) in the conversion of at least one amino sugar, preferably 2-amino-2-deoxysugar, into fructose and ammonia.

According to a preferred embodiment, the amino sugar is obtained from hydrolyzing biomaterials, preferably chitin-or chitosan-containing biomaterials, such as shellfish exoskeletons, insects or fungi.

In a further aspect, the present invention is directed to a composition comprising ammonia and fructose obtained by a process for converting amino sugar to fructose and ammonia using one enzyme.

According to a preferred embodiment, said composition comprising ammonia and fructose does not contain phosphate, neither in free nor conjugated form.

### Experimental Part

### Analytical Methods

### Ammonia Determination in Enzymatic Transformations

Reaction mixtures (50 μL) were diluted with a 1.5% (w/V) NaOH solution (930μL). The ammonium electrode (DX218-NH4, Mettler Toledo) was then immersed into the sample until a stable reading is obtained (typically 2 min). The values were then compared with the calibration curve of ammonium standards of known concentrations between 10 μM and 1 mM.

### Fructose and Glucosamine Determination in Enzymatic Transformations using TLC.

One microliter of the enzyme reaction mixture was spotted onto a silica 60 F254TLC plate (Merck), and the plate contents were developed using a solvent mixture of acetonitrile/acetic acid/water (7:1.5:1.5, V/V/V). The developed plate contents were dried and visualized using a solvent mixture of acetone, phosphoric acid, aniline and diphenylamine (90:8:2:2, V/V/V/w), and then subjected to heating for 10 min at 120° C.

Fructose spots were observed at Rf=0.61. Glucosamine spots were observed at Rf=0.31.

### Sample Derivatization for Photometric Measurement of Fructose in Enzymatic Transformations (based on Agri. Biol. Chem. (1968) 32:6, 689-706).

Reaction mixtures (50 μL) were centrifuged at 12000 g for 10 min at room temperature, and 40 μL samples of the supernatant were taken and mixed with 35 μL conc. HCl and 5 μL of resorcinol-thiourea reagent (consisting of 0.1% (w/V) resorcinol and 0.25% (w/V) in glacial acetic acid). The mixed samples were incubated at 80° C. for 10 min and then centrifuged at 12000 g for 10 min at room temperature. The supernatants (50 μL) were then transferred to 96 microplate and the absorbance at λ=505 nm measured with a microplate photometer (Thermo Multiskan).

### Activity test using Seliwanoff's derivatization method of Fructose

### (Photometric Resorcinol Test at μ=505 nm)

### Materials

Unless otherwise indicated, all materials were obtained from commercial sources.

### Glucosamine

Glucosamine can be obtained commercially in the form of glucosamine hydrochloride. Alternatively, glucosamine may be obtained via hydrolysis of chitin, chitosan or N-acetylglucosamine. Likewise, glucosamine may be obtained via enzymatic conversion of N-acetylglucosamine.

### Hydrolysis of chitin, chitosan or N-acetylglucosamine to glucosamine

Glucosamine can be prepared from chitin, chitosan, or N-acetylglucosamine using hydrochloric acid (Ainbu, A. et al. (2008) Biomacromolecules, 9, 1870-1875). Powdered chitin from shrimp shells (1 g) is suspended in 1 mL of dilute HCl (100 mM) and wettened and suspended in 50 mL of a 6 M HCl solution. This suspension is heated for 3 h at 100° C. and then cooled down to room temperature. The solution is adjusted to pH 7 via dropwise addition of NaOH solution (4 M) and dried using rotary evaporation.

Enzymatic conversion of N-acetylglucosamine to glucosamine

Glucosamine can be also produced enzymatically from N-acetylglucosamine (Lv, Y. M., et al. (2017) Green Chem., 19, 527-535): 50 g/L of N-acetylglucosamine are incubated at 37° C. in the presence of 120 mg/L of Cyclobacterium marinum N-acetylglucosamine deacetylase in 200 mM sodium phosphate buffer (pH 8.0) for 12 h. After completion of the reaction, the reaction mixture is dried using rotary evaporation.

### Preparation of the GPDA Enzyme:

Genes encoding the wild-type or mutant variants of GDPA were synthesized in an E. coli codon-optimized form (Genscript) and delivered in a pUC19 vector with ampicillin resistance. The protein-encoding insert was cut via Ndel and Xhol restriction enzymes (Takara) and ligated in a pET30a expression vector via T4Ligase (Takara). The GPDA enzymes were prepared as follows: a fresh single colony from an LB agar plate supplemented with 50 mg/mL Kanamycin and used to grow E. coli strain BL21 comprising the respective plasmid was used to inoculate 5 mL of LB liquid medium with the same concentration of Kanamycin. Following incubation of said culture over night at 120 rpm and 37° C. 2 mL of the overnight culture was used to inoculate 400 mL of the same medium in a 2 L flask. The flask was shaken at 37° C. and 120 rpm until an OD600 of 0.6 was reached, i.e. approximately two to four hours. The expression of the respective enzyme was subsequently induced by addition of 0.4 mL IPTG (Isopropyl-ß-D-thiogalactopyranoside, 238 mg/mL), followed by incubation over night at 18° C. and 120 rpm under vigorous shaking. Cells were then pelleted at 4000 g and 4° C. for 20 minutes. The supernatant was disposed of, the pellet was resuspended in phosphate buffer (50 mM NaH₂PO₄, 300 mM NaCl, 10 mM Imidazole) and lysed using ultrasonication (1 s pulse followed by incubation for 4 s, repeated for a total of 20 minutes). The cell lysate was centrifuged (18000 g, 20 min, 4° C.), and applied to a His-trap column having a bed volume of 5 mL. The His-trap column was washed using phosphate buffer (50 mM NaH₂PO₄, 300 mM NaCl) comprising 20 mM imidazole. The GPDA enzyme was eluted by application of 10mL of the same buffer comprising 250 mM imidazole. Fractions comprising the GPDA enzyme were pooled and then stored at −80° C., dialyzed against phosphate buffer to remove imidazole and concentrated via Vivaspin tubes and centrifugation at 4° C.

Further details regarding GPDA variants used in the examples and comparative examples are given in Table 1 below. Unless otherwise indicated, all enzymes are obtained as wild type variants from the respective organism of origin. Regarding the enzymes denoted as Es1/Es2, Ph1/Ph2/Ph3, BI1/B12 and Sn1/Sn2, it is pointed out that they are homologs in the same organism of origin (descended from the same enzyme family, but probably having different physiological functions in the organism) and exclusively ‘wild-type’ variants.

TABLE 1

| Sample | | | |
|---|---|---|---|
| No° | Identifier | GPDA Origin | SEQ ID NO |
| 1 | Ef | Enterococcus faecalis | 1 (AA)/2 (DNA) |
| 2 | Es1 | Exiguobacterium sibiricum | 3 (AA)/4 (DNA) |
| 3 | Es2 | Exiguobacterium sibiricum | 5 (AA)/6 (DNA) |
| 4 | Cn | Clostridium nexile | 7 (AA)/8 (DNA) |
| 5 | Df | Dyadobacter fermentans | 9 (AA)/10 (DNA) |
| 6 | Ph1 | Pedobacter heparinus | 11 (AA)/12 (DNA) |
| 7 | Ph2 | Pedobacter heparinus | 13 (AA)/14 (DNA) |
| 8 | Ph3 | Pedobacter heparinus | 15 (AA)/16 (DNA) |
| 9 | Gt | Granulicella tundricola | 17 (AA)/18 (DNA) |
| 10 | Tr | Terriglobus roseus | 19 (AA)/20 (DNA) |
| 11 | Am | Akkermansia muciniphila | 21 (AA)/22 (DNA) |
| 12 | Bl1 | Bifidobacterium longum | 23 (AA)/24 (DNA) |
| 13 | Bl2 | Bifidobacterium longum | 25 (AA)/26 (DNA) |
| 14 | Mx | Myxobacterium xanthis | 27 (AA)/28 (DNA) |
| 15 | Sn1 | Stackebrandtia nassauensis | 29 (AA)/30 (DNA) |
| 16 | Sn2 | Stackebrandtia nassauensis | 31 (AA)/32 (DNA) |
| 17 | Ef- | Mutant variant - derived from | 33 (AA)/34 (DNA) |
| | Ser74Ala | Enterococcus faecali | |
| 18 | Ef- | Mutant variant - derived from | 35 (AA)/36 (DNA) |
| | Thr144Ala | Enterococcus faecalis | |

### Working Examples

### Enzymatic Generation of Ammonia by Ef-GPDA:

The enzymatic reaction mixtures consisting of 30 μL of purified Ef-GPDA solution (1.8 mg/mL), 35 μL of MES buffer (500 mM, pH 7.0), 5 μL of MgCl₂ (30 mM), and 30 μL of glucosamine (concentration range between 3 and 140 mM). Samples were incubated at 40° C. for 10 h. The ammonia concentration was measured potentiometrically using an ammonia gas sensing electrode.

TABLE 2

| | Glucosamine Conc. [mM] | | | | | | | |
|---|---|---|---|---|---|---|---|---|
| | 1 | 2 | 3 | 5 | 6 | 8 | 10 | 15 |
| NH₃ Conc. | 0.45 | 0.59 | 0.69 | 0.98 | 1.32 | 1.50 | 2.01 | 2.60 |
| [mM] | | | | | | | | |

### Activity of Ef-GPDA at Different pH Values:

Enzymatic reaction mixtures consisting of 30 μL of purified Ef-GPDA solution (1.8 mg/mL), 5 μL of MgCl₂ (30 mM), and 30 μL of glucosamine (15 mM) and 35 μL of the following buffer solutions:

Tris/HCl buffer (500 mM) for pH values 4.0, 5.0, 6.0, 7.0, and 8.0,Bicine buffer (500 mM) for pH 9.0, and Carbonate/Bicarbonate buffer (500 mM) for pH 10.Samples were incubated at 40° C. for 10 h. The conversion of glucosamine to fructose was measured photometrically at λ=505 nm after samples were derivatized with resorcinol.

TABLE 3

| pH | 4.0 | 5.0 | 6.0 | 7.0 | 8.0 | 9.0 | 10.0 |
|---|---|---|---|---|---|---|---|
| Glucosamine to Fructose | 6.8 | 6.8 | 5.5 | 12.8 | 9.4 | 3.0 | 3.5 |
| Conversion [%] | | | | | | | |

### Activity of Ef-GPDA at Different Temperatures:

The enzymatic reaction mixtures consisting of 30 μL of purified Ef-GPDA solution (1.8 mg/mL), 35 μL of MES buffer (500 mM, pH 7.0), 5 μL of MgCl₂ (30 mM), and 30 uL of glucosamine (15 mM). Samples were incubated at various temperatures between 4° C. and 50° C. for 6 h. The conversion of glucosamine to fructose was measured photometrically at λ=505 nm after samples were derivatized with resorcinol.

TABLE 4

| Temperature [° C.] | 4 | 10 | 16 | 20 | 25 | 30 | 37 | 42 | 50 |
|---|---|---|---|---|---|---|---|---|---|
| Glucosamine to Fructose | 2.1 | 1.4 | 2.6 | 0.9 | 4.2 | 5.2 | 8.2 | 5.3 | 0.4 |
| Conversion [%] | | | | | | | | | |

### Temperature stability of Ef-GPDA:

The temperature stability of Ef-GPDA was determined by incubating the enzyme at 4° C., 30° C., 37° C., 42° C., and 50° C. for periods of 0 h, 2 h, 4 h, 6 h, 12 h, 24 h, and 48 h. Then, these enzyme solutions were used in enzymatic reaction mixtures consisting of 30 μL of purified Ef-GPDA solution (1.8 mg/mL), 35 μL of MES buffer (500 mM, pH 7.0), 5 μL of MgCl2 (30 mM), and 30 μL of glucosamine (concentration range between 3 and 140 mM). Samples were incubated at 40° C. for 6 h. The conversion of glucosamine to fructose was measured photometrically at λ=505 nm after samples were derivatized with resorcinol.

TABLE 5

| | | Time [h] | | | | | | |
|---|---|---|---|---|---|---|---|---|
| | | 0 | 2 | 4 | 6 | 12 | 24 | 48 |
| Glucosamine to | T = 4° C. | 10.15 | 9.87 | 9.78 | 9.67 | 8.64 | 9.36 | 7.49 |
| Fructose Conversion | T = 30° C. | 10.15 | 9.37 | 8.38 | 8.42 | 9.11 | 8.20 | 8.95 |
| [%] | T = 37° C. | 10.15 | 8.64 | 8.76 | 8.42 | 8.64 | 6.97 | 7.49 |
| | T = 42° C. | 10.15 | 6.75 | 4.33 | 4.08 | 3.70 | 0.80 | 1.44 |
| | T = 50° C. | 10.15 | 4.02 | 3.17 | 2.01 | 1.17 | 0.80 | N/A |

### Kinetic Parameters of Ef-GPDA:

For the kinetic analysis enzymatic reaction mixtures consisting of 30 μL of purified Ef-GPDA solution (1.8 mg/mL), 35 μL of MES buffer (500 mM, pH 7.0), 5 μL of MgCl₂ (30 mM), and 30 μL of glucosamine (concentration range between 3 and 140 mM). Samples were incubated at 40° C. for 10 h. The conversion of glucosamine to fructose was measured photometrically at λ=505 nm after samples were derivatized with resorcinol.

Vmax=4.0±0.2 μmol/min

Km=21.8±2.3 mM

TABLE 6

| | Glucosamine Conc. [mM] | | | | | | |
|---|---|---|---|---|---|---|---|
| | 1 | 5 | 10 | 15 | 25 | 35 | 45 |
| Reaction | 0.17 | 0.72 | 1.35 | 1.74 | 1.98 | 2.45 | 2.68 |
| Velocity [μM/min] | | | | | | | |

### Timecourse Experiment to Determine The Activity of Ef-GPDA:

Enzymatic reaction mixtures consisting of 30 μL of purified Ef-GPDA solution (1.8 mg/mL), 35 μL of MES buffer (500 mM, pH 7.0), 5 uL of MgCl₂ (30 mM), and 30 μL of glucosamine (15 mM). Samples were incubated at 40° C. for up to 24 h.

The conversion of glucosamine to fructose was measured photometrically at λ=505 nm after samples were derivatized with resorcinol.

TABLE 7

| Time of Enzymatic Reaction [h] | 3.5 | 6 | 12 | 17 | 23 |
|---|---|---|---|---|---|
| Glucosamine to Fructose | 2.51 | 3.44 | 8.24 | 10.57 | 16.84 |
| Conversion [%] | | | | | |

### Comparison of the Activity of various GPDA Genes Towards Glucosamine:

Enzymatic reaction mixtures consisting of 30 μL of purified GPDA protein variants (1.8 mg/mL), 35 μL of MES buffer (500 mM, pH 7.0), 5 μL of MgCl₂ (30 mM), and 30 μL of glucosamine (15 mM). Samples were incubated at 40° C. for 10 h. The conversion rate of glucosamine to fructose was measured photometrically at A=505 nm after samples were derivatized with resorcinol.

TABLE 8

| GPDA - Enzyme Variant | Es1 | Cn | Df | Ph1 | Ph2 | Ph3 | Sn1 | Gt |
|---|---|---|---|---|---|---|---|---|
| Relative conversion | 5.4 | 0.0 | 0.0 | 0.0 | 0.7 | 3.4 | 1.3 | 5.4 |
| rate [%] | | | | | | | | |

| GPDA - Enzyme Variant | Ef | Sn2 | Es2 | Tr | Am | Bl1 | Mx | Bl2 |
|---|---|---|---|---|---|---|---|---|
| Relative conversion | 100 | 1.3 | 0.0 | 0.0 | 0.7 | 3.4 | 4.0 | 0.0 |
| rate [%] | | | | | | | | |

GPDA Origin:

Es1, Es2: Exiguobacterium sibiricum; Cn: Clostridium nexile; Df: Dyadobacter fermentans; Ph1, Ph2, Ph3: Pedobacter heparinus; Gt: Granulicella tundricola; Ef: Enterococcus faecalis; Tr: Terriglobus roseus; Am: Akkermansia muciniphila; BI1, BI2: Bifidobacterium longum; Mx: Myxobacterium xanthis; Sn1,Sn2: Stackebrandtia nassauensis.

### Ef-GPDA Wild-Type and Mutant Variants

Ef-GPDA mutant variants were generated and tested for enzymatic activity following the same test protocol as detailed above for GPDA variants originating from different organisms.

The mutant variants of Ef-GPDA were selected based on studies of the glucosamine-6-phosphate deaminase family (i.e from the E. coli variant).

TABLE 9

| Ef-GPDA Enzyme Variant | Ser74Ala | Thr144Ala | Wild Type |
|---|---|---|---|
| Relative Enzymatic Activity [%] | 107.7 | 103.5 | 100 |

1. A process for converting amino sugar to fructose and ammonia using one enzyme.

2. The process according to claim 1, wherein the enzyme is a hydrolase, preferably glucosamine-6-phosphate deaminase (GPDA enzyme), more preferably Ef-GPDA from Enterococcus faecalis.

3. The process according to claim 2, wherein the hydrolase, preferably glucosamine-6-phosphate deaminase (GPDA enzyme) is an isolated polypeptide or a whole-cell biocatalyst.

4. The process according to claim 1, wherein at least 10%, preferably at least 15% of the amino sugar are converted to fructose and ammonia after 24 h of enzymatic conversion.

5. The process according to claim 1, wherein the amino sugar is an amino monosaccharide, preferably a 2-amino-2-deoxysugar and more preferably is glucosamine.

6. The process according to claim 1, wherein the process is a one-step process.

7. The process according to claim 1, wherein the amino sugar is obtained from hydrolyzing biomaterials, preferably chitin-or chitosan-containing biomaterials, such as shellfish exoskeletons, insects or fungi.

8. The process according to claim 1, wherein in said step the glucosamine is contacted with one enzyme in the presence of water.

9. The process according to claim 1, wherein the enzyme, preferably the hydrolase, more preferably the glucosamine-6-phosphate deaminase (GPDA enzyme), is immobilized.

10. The process according to claim 1, comprising the steps of:
optionally hydrolyzing a biomaterial, preferably a biomaterial containing amino sugars, preferably containing amino polysaccharides, more preferably containing chitin and/or chitosan;
converting an amino sugar, preferably an amino monosaccharide, to fructose and ammonia using one enzyme, preferably using a hydrolase, more preferably a glucosamine 6-phosphate deaminase.

11. The process according to claim 1, wherein the enzymatically converting is a deamination-isomerization reaction.

12. An enzyme for converting amino sugar, preferably amino monosaccharide, preferably 2-amino-2-deoxysugar and more preferably glucosamine to fructose and ammonia.

13. Enzyme according to claim 12, wherein the enzyme is a hydrolase, preferably glucosamine-6-phosphate deaminase (GPDA enzyme), more preferably glucosamine-6-phosphate deaminase (GPDA enzyme) comprising an amino acid sequence that is at least 80%, preferably at least 90%, more preferably at least 95% and even more preferably at least 98.5% identical with the sequence according to SEQ ID NO: 1.

14. Use of an enzyme according to claim 12 in the conversion of at least one amino sugar, preferably 2-amino-2-deoxysugar, into fructose and ammonia, preferably the amino sugar is obtained from hydrolyzing biomaterials, preferably chitin-or chitosan-containing biomaterials, such as shellfish exoskeletons, insects or fungi.

15. Composition comprising ammonia and fructose obtained by a process according to claim 1.

16. Composition for converting amino sugar to fructose and ammonia comprising
water;
an amino sugar, preferably an amino monosaccharide, more preferably a 2-amino-2-deoxysugar and even more preferably glucosamine;
a hydrolase, preferably a glucosamine-6-phosphate deaminase (GPDA enzyme), more preferably glucosamine-6-phosphate deaminase (GPDA enzyme) with an amino acid sequence that is at least 80%, preferably at least 90%, more preferably at least 95% and even more preferably at least 98.5% identical with the sequence according to SEQ ID NO: 1.
